(19) United States
(12) Patent Application Publication
(10) Pub. No.: US 2024/0335389 A1
(43) Pub. Date: Oct. 10, 2024

(54) PLANT-BASED HARD CAPSULE FOR RAPID DISINTEGRATION AND METHOD THEREOF

(71) Applicant: HUAQIAO UNIVERSITY, Quanzhou (CN)

(72) Inventors: Meitian XIAO, Quanzhou (CN); Chuqi He, Quanzhou (CN); Chaoxing Chen, Shunchang (CN); Jing Ye, Quanzhou (CN); Shipeng Chen, Shunchang (CN); Yucheng Yang, Quanzhou (CN); Yayan Huang, Quanzhou (CN); Bingde Zheng, Quanzhou (CN); Xueqin Zhang, Quanzhou (CN); Na Zhang, Quanzhou (CN)

(21) Appl. No.: 18/131,908

(22) Filed: Apr. 7, 2023

(51) Int. Cl. A61K 9/48; A61K 47/38

(52) U.S. Cl. CPC A61K 9/4816; A61K 47/38

(57) ABSTRACT

A method for preparing a plant-based hard capsule for rapid disintegration, the method comprises: weighing a coagulant aid, dissolving in distilled water at 75-90° C., and stirring to obtain a first solution; maintaining a temperature of the first solution at 75-90° C., adding a gel-forming agent, and stirring; adding a film-forming agent with a weight percentage of 5%-25%, stirring, finally adding a plasticizer by a weight percentage of 0-3%, and stirring until the film-forming agent and the plasticizer are completely dissolved to obtain a material solution; leaving the material solution to stand at 45-60° C. to obtain a gel solution; and cooling the gel solution to 40-50° C., dipping the gel solution for forming, and drying for 100-200 minutes under a condition of 20-35° C. and a 40%-60% relative humidity; and demoulding, cutting, and combining together by sleeving to obtain the plant-based hard capsule for the rapid disintegration.

### FIELD OF THE DISCLOSURE

The present disclosure relates to a field of biopharmaceutical preparation, and in particular relates to a plant-based hard capsule (i.e., a plant-based pharmaceutical hard capsule) for rapid disintegration and a method thereof.

### BACKGROUND OF THE DISCLOSURE

Hard capsules can cover up unpleasant tastes and smells of many drugs to decrease stomach irritation. Therefore, capsules are widely used in the production of various solid preparations. In general, the substrate of pharmaceutical capsules usually is gelatin. However, defects and deficiencies of gelatin-based hard capsules are increasingly obvious. First, gelatin is a protein substance, and its strong hygroscopic properties can induce enclosed drugs to absorb moisture, resulting in a shortened period of validity and unqualified quality of drugs. Second, under the prolonged storage time, a cross-linking reaction will occur between the gelatin and aldehydes or excipients, so that the disintegration time of the capsules is prolonged and drugs cannot be timely released in the stomach. In addition, gelatin-based capsules are greatly rejected due to the influence of mad cow disease and poison capsules. Therefore, the development of novel materials for capsules has become a research hotspot.

Therefore, plant-based hard capsules have emerged. The film-forming agent, as a main component, is combined with other gel-forming agents, coagulation aids, plasticizers, etc. to form a prescription to prepare the plant-based hard capsules. Compared with traditional gelatin-based hard capsules, the plant-based hard capsules are safer and more stable. The existing plant-based hard capsules (e.g., a plant-based pharmaceutical hard capsule or a plant-based pharmaceutical hollow capsule) are mostly Pullulan-based hard capsules. Hydroxypropyl methylcellulose based (HPMC-based) hard capsules using hydroxypropyl methylcellulose as the main material have lower production cost than the Pullulan-based hard capsules. The HPMC-based hard capsules have low water content and good flexibility, and the brittle breaking phenomenon will not occur after long-term storage. Carrageenan, usually used as a gel-forming agent for the plant-based hard capsules, is considered as a new capsule material with a good development prospect because of not only a wide and safe source of raw materials but also good gelling properties.

At present, the HPMC-based hard capsules and Pullulan-based hard capsules added with the gel-forming agent and the coagulation aids on the market have a poor dissolubility and a slow disintegration time, which are basically used in health foods. In the test of disintegration time, the disintegration time of most plant-based hard capsules prepared using carrageenan as the gel-forming agent is faster in a pH 1.2 hydrochloric acid solution or deionized water, but the disintegration time will be prolonged in a pH 4.5 phosphate buffer solution or a pH 6.8 phosphate buffer solution and may take more than 1 hour to be completely dissolved. In contrast, the disintegration time of the plant-based hard capsules using Gellan gum as the gel-forming agent will be slightly shortened in the pH 4.5 phosphate buffer solution, the pH 6.8 phosphate buffer solution, or the deionized water, but the disintegration will be prolonged in the pH 1.2 hydrochloric acid solution and may take more than 1 hour to be completely dissolved. At present, there is no plant-based hard capsule using a gel-forming agent on the market that can be rapidly disintegrated in the following four media at the same time: the pH 1.2 hydrochloric acid solution, the pH 4.5 phosphate buffer solution, the deionized water, and the pH 6.8 phosphate buffer solution. Plant-based hard capsules on the market basically cannot meet the time limit on the disintegration of gelatin-based capsules (less than 10 minutes) required by the test of the time limit on the disintegration in paragraph 0921, section 4 of the Pharmacopoeia of the People's Republic of China, 2020 edition and cannot meet rapid disintegration requirements of capsules for medicinal use. In a dissolution test, the hard capsules for medicinal use should meet a dissolution standard of 30 minutes. The time for breaking shells of the plant-based hard capsules on the market usually is about 10 minutes, and the complete dissolution time of the plant-based hard capsules is greater than 30 minutes.

### BRIEF SUMMARY OF THE DISCLOSURE

The present disclosure provides a plant-based hard capsule (i.e., a plant-based pharmaceutical hard capsule, a plant-based hollow capsule for medical use, or a plant-based pharmaceutical hollow capsule) for rapid disintegration and a method thereof to overcome the deficiencies of the existing techniques.

In order to solve the aforementioned technical problems, the present disclosure provides a method for preparing the plant-based pharmaceutical hard capsule for rapid disintegration, the method comprises the following steps:
(1) weighing coagulant aids with a weight percentage of 0.01%-3%, dissolving in distilled water at 75-90° C., and stirring until the coagulant aids are completely dissolved to obtain a first solution; maintaining a temperature of the first solution at 75-90° C., adding (e.g., slowly adding) a gel-forming agent with a weight percentage of 0.1%-5%, and stirring until the gel-forming agent is completely dissolved to avoid agglomeration caused by swelling due to a large amount of the gel-forming agent meeting water, resulting in prolongation of a dissolution time; adding (e.g., adding in batches) a film-forming agent with a weight percentage of 5%-25%, stirring for 2-5 hours, finally adding a plasticizer with a weight percentage of 0-3%, and stirring until the film-forming agent and the plasticizer are completely dissolved to obtain a material solution;
(2) leaving the material solution obtained in the step (1) to stand at 45-60° C. for 8-12 hours to obtain a stable gel solution, and stirring the stable gel solution to avoid the stable gel solution from being divided into layers; and
(3) cooling the stable gel solution obtained in the step (2) to 40-50° C., dipping into the stable gel solution for forming, then drying for 100-200 minutes under a condition of 20-35° C. and a 40%-60% relative humidity to obtain a crude product of a capsule cap and a capsule body; and demoulding, cutting, and combining together by sleeving to obtain the plant-based pharmaceutical hard capsule.

With respect to the step 1, the sequence of adding the coagulant aids first and then the gel-forming agent can avoid an agglomeration of a contact part between the coagulant aids and the gel-forming agent when adding the coagulant aids later.

With respect to the step 1, the temperature is maintained at 75-90° C., a dissolution time of the coagulant aids, the gel-forming agent, and the film-forming agent is appropriately sped up, and the viscosity of the compound gel solution is reduced. The temperature is conducive to uniform dispersion and complete dissolution of the material solution. When the temperature is too high, a partial chain breakage in a molecular chain of polysaccharides will occur when long-term stirring takes place at a higher temperature. When the temperature is too low, the gel-forming agent is slowly dissolved, thus prolonging the dissolution time and increasing energy consumption.

In a preferred embodiment, the film-forming agent comprises at least one of hydroxypropyl methylcellulose (HPMC), sodium carboxymethyl starch, or hydroxypropyl starch.

HPMC is a good film-forming material, but HPMC does not have a gelatinous property by itself. Only a certain amount of the gel-forming agent is added, and a HPMC solution can be dipped for forming. Through studies, it is found that sodium carboxymethyl starch not only has strong water absorption and expansibility, but can be also used as a high-efficiency disintegrating agent for plant-based hard capsules, so that the corresponding plant-based hard capsules are rapidly disintegrated. Therefore, in the present disclosure, the appropriate sodium carboxymethyl starch can be added to speed up the disintegration time.

In a preferred embodiment, the gel-forming agent comprises at least one of Gelan gum, ι-Carrageenan, or κ-carrageenan.

An addition of the coagulant aids can help colloids with gelation abilities (e.g., the gel-forming agent) to quickly form a gel. The inorganic salt ions of the coagulant aids mediate to act as a “salt bridge” with the colloids, then the coagulant aids and the colloids are piled up side by side, and are then entangled with each other to form the gel, thus effectively improving gelation effects of the gel-forming agent. The carrageenan has characteristics of gelation, film forming, stable dispersion, etc. to form hydrophilic colloids and is a good material for plant-based hard capsules. With respect to the κ-Carrageenan and the gellan gum, potassium ion can be selected as the coagulant aid, and a compound ratio of the gel-forming agent and the coagulant aids can range from 3:0.1 to 2:0.3. With respect to the ι-Carrageenan, calcium ion or sodium ion can be selected as the coagulant aids, and the compound ratio of the gel-forming agent and the coagulant aid can range from 3:0.1 to 1:0.3. When strength of the gel needs to increase and a film-forming speed needs to be quickened, citrate ion can be selected as the coagulant aid. When the disintegration time of the plant-based hard capsules needs to be accelerated, chloride ion can be selected as the coagulant aid. The compound ratio of the gel-forming agent and the coagulant aid can range from 2.5 to 10.0. When an amount of the coagulant aid used is larger, the disintegration time of the plant-based hard capsules in four buffer solution media will be prolonged, and the requirements for rapid disintegration cannot be met. When the amount of the coagulant aid used is smaller, it will lead to a difficulty in gelation for the compound gel solution and is not suitable for industrial production.

In a preferred embodiment, the coagulant aid comprises at least one of potassium citrate, sodium citrate, potassium chloride, sodium chloride, or calcium chloride.

The coagulant aid can accelerate the gelation process of the carrageenan, and different kinds of carrageenan have different sensitivities to different ions. The κ-carrageenan and potassium ions can form a hard and brittle gel. The mechanical properties of the plant-based hard capsules prepared by the κ-carrageenan and the potassium ions are relatively good, while the disintegration time of the plant-based hard capsules in a pH 4.5 phosphate buffer solution or a pH 6.8 phosphate buffer solution is longer. The ι-carrageenan acts with calcium or sodium ions to form a soft gel. The soft gel has excellent water retention ability, elasticity, thixotropy, and stability against thaw and has better disintegration properties. However, the mechanical properties of the plant-based hard capsules prepared only using the ι-carrageenan gel and the calcium or sodium ions as the gel-forming agent are poor. The κ-Carrageenan and the ι-Carrageenan are combined as the gel-forming agent to effectively combine their advantages and to compensate for their deficiencies.

Therefore, given comprehensive consideration of the disintegration time and physical and chemical properties, the present disclosure adopts different types and different proportions of coagulant aids according to different gel-forming agents to achieve rapid disintegration and to solve a technical problem of the long disintegration time of the plant-based hard capsules using the carrageenan as the main gel-forming agent.

In a preferred embodiment, the plasticizer comprises at least one of glycerol, polyethylene glycol (PEG), or sorbitol.

In a preferred embodiment, the gel-forming agent is ι-Carrageenan or κ-Carrageenan.

In a preferred embodiment, the coagulant aid comprises potassium chloride or potassium citrate and at least one of sodium citrate, sodium chloride, or calcium chloride.

In a preferred embodiment, the film-forming agent is hydroxypropyl methylcellulose.

In a preferred embodiment, a weight ratio of the film-forming agent, the gel-forming agent, the coagulant aid, the plasticizer, and the distilled water is (6.41-16.39):(0.76-1.83):(0.13-0.31):(0-1.33):(81.95-91.53).

In a preferred embodiment, the film-forming agent is the HPMC, the gel-forming agent is the κ-Carrageenan and the ι-Carrageenan, the plasticizer is the glycerin, and the coagulant aid is the calcium chloride and the potassium chloride.

A weight ratio of the HPMC, the κ-Carrageenan, the ι-Carrageenan, the glycerin, the calcium chloride, the potassium chloride, and the distilled water is 16.39:0.41:0.61:0.2:0.02:81.95.

The present disclosure provides a plant-based hard capsule for rapid disintegration prepared by the aforementioned method.

The present disclosure provides a plant-based hard capsule for rapid disintegration prepared by the aforementioned method.

In conclusion, the present disclosure has at least one of the following advantages.
1. Compared with the existing techniques, the plant-based pharmaceutical hard capsule for the rapid disintegration prepared by the present disclosure can enable the plant-based hard capsules prepared by different materials to show rapid disintegration characteristics in a pH 1.2 hydrochloric acid solution, a pH 4.5 phosphate buffer solution, deionized water, or a pH 6.8 phosphate buffer solution. A release of an agent of the plant-based hard capsule can be completely guaranteed.
2. A problem of a long disintegration time and a slow dissolution rate in the HPMC-based capsules caused by adding the carrageenan as a gel-forming agent is solved by compounding the carrageenan and the inorganic salt ions (i.e., cations and anions).
3. The process of the present disclosure is simple, can be directly applied to mature production equipment for gelatin-based capsules, and has potential in industrial production.
4. The product prepared by the present disclosure fully satisfies the requirements for a capsule shell of the hard capsule in the Pharmacopoeia of the People's Republic of China: a wall thickness is 0.11±0.02 mm, a drying loss is <8%, the disintegration time is <15 minutes, a fragility and a tightness are both 0, an ignition residue is <4%, and a heavy metal content is <20 ppm.
5. The present disclosure can avoid potential safety risks caused by a source and a treatment of the gelatin capsule not being up to a standard. The present disclosure has a good application value in the field of medical products and health products with high health-safety requirements.

Other features and beneficial effects of the present disclosure will be described in the following specification, will partially become obvious from the specification, or will be understood by implementing the present disclosure. An objective and other beneficial effects of the present disclosure can be achieved and obtained through a structure specifically illustrated in the specification, the claims, and the drawings.

Therefore, a development of the plant-based pharmaceutical hard capsule using HPMC as a main film-forming agent, using the carrageenan as a main gel-forming agent, having a simple process, and being rapidly disintegrated has practical positive significance for a rapid development of the pharmaceutical industry.

### BRIEF DESCRIPTION OF THE DRAWINGS

In order to explain the embodiments of the present disclosure or the technical solutions in the prior art more clearly, the drawings needed in the description of the embodiments or the prior art will be briefly described below. It is obvious that the drawings in the following description are merely some embodiments of the present disclosure, and other drawings can also be obtained from these drawings without creative work for ordinary skill in the art. In the following description, the positional relationships described in the drawings, unless otherwise specified, are based on directions that components are illustrated in the drawings.

FIG. 1 illustrates test results of a plant-based hard capsule for rapid disintegration prepared in Embodiments 1-10 of the present disclosure.

### DETAILED DESCRIPTION OF THE EMBODIMENTS

In order to make an objective, a technical solution, and advantages of the embodiments of the present disclosure clearer, the technical solution in the embodiments of the present disclosure will be clearly and completely described in combination with the drawings in the embodiments of the present disclosure. It is obvious that the described embodiments are merely some embodiments of the present disclosure, while not all embodiments of the present disclosure. Designed technical features in different embodiments of the present disclosure described below can be combined with each other provided that no conflict exists between the designed technical features. It is intended that the present disclosure cover all other embodiments provided they are made based on the embodiments presently presented in the present disclosure and without creative work by ordinary skill in the art to which the present disclosure belongs.

In the description of the present disclosure, it should be noted that all terms (including technical terms and scientific terms) used in the present disclosure have a same meaning as a meaning of all terms commonly understood by those of ordinary skill in the art, and should not be understood as limitations of the present disclosure. It should be further understood that the terms used in the present disclosure should be understood to have a meaning consistent with a meaning of the terms in a context of the present specification and related fields and should not be understood in an idealized way or an overly formal sense, unless clearly defined in the present disclosure.

Although a numerical range and a parameter listed in a wide range of the present disclosure are approximate values, a record of a numerical value listed in a specific embodiment should be as accurate as possible. However, any numerical value basically has a certain error, and the error is an inevitable result of a standard deviation obtained in a corresponding measurement method.

A same reference number and/or a same mark may be repeatedly used in different embodiments disclosed below. The repetition is used for simplification and clarity of the present disclosure and is not intended to limit a specific relationship between the different embodiments and/or structures discussed in the present disclosure.

The technical solution of the present disclosure is further explained and described by specific embodiments.

### Embodiment 1

(1) Each raw material is weighted according to the following weight percentages:

| | |
|---|---|
| Hydroxypropyl methylcellulose | 12.83% |
| κ-Carrageenan | 0.43% |
| ι-Carrageenan | 0.43% |
| Glycerin | 0.64% |
| Sodium chloride | 0.10% |
| Potassium chloride | 0.03% |
| Distilled water | 85.54% |

(2) The sodium chloride, the potassium chloride, and the glycerin are dissolved in the distilled water at a normal temperature (e.g., 20° C.-30° C.) and then heated to 80° C. The κ-Carrageenan and the ι-Carrageenan are slowly added in sequence and are stirred at a constant temperature of 80° C. for 1.5 hours until the κ-Carrageenan and the ι-Carrageenan are completely dissolved. The hydroxypropyl methylcellulose is then added and stirred at a constant temperature of 80° C. for 2.5 hours until the hydroxypropyl methylcellulose is completely dissolved, and glycerin is finally added and stirred to even to obtain a material solution. In this step, an appropriate amount of an edible pigment can be added as a colorant according to the situation.
(3) The material solution obtained in step (2) is left to stand at 60° C. for stable heat retention for 8 hours to obtain a stable gel solution.
(4) The stable gel solution is cooled to 46° C., dipped using a mold, flipped upward and downward for forming for 6 seconds, and dried in an oven for 150 minutes at 30° C. and relative humidity (RH) 50% to obtain a crude product of a capsule cap and a capsule body. The crude product of the capsule cap and the capsule body is released from the mold by a mold release device, cut, and then combined together by sleeving to obtain a hard capsule, that is, a plant-based hard capsule (e.g., a plant-based pharmaceutical hard capsule) for rapid disintegration.

### Embodiment 2

(1) Each raw material is weighted according to the following weight percentages:

| | |
|---|---|
| Hydroxypropyl methylcellulose | 12.69% |
| Sodium carboxymethyl starch | 0.85% |
| κ-Carrageenan | 0.17% |
| ι-Carrageenan | 0.68% |
| Glycerin | 0.85% |
| Sodium chloride | 0.14% |
| Distilled water | 84.62% |

(2) The sodium chloride and the glycerin are dissolved in the distilled water at a normal temperature (e.g., 20° C.-30° C.) and then heated to 80° C. The κ-Carrageenan and the ι-Carrageenan are added in sequence and stirred at a constant temperature of 80° C. for 1.5 hours until the κ-Carrageenan and the ι-Carrageenan are completely dissolved. The hydroxypropyl methylcellulose and the sodium carboxymethyl starch are then slowly added at a constant temperature of 80° C. for 2.5 hours until the hydroxypropyl methylcellulose and the sodium carboxymethyl are completely dissolved to obtain a material solution. In this step, an appropriate amount of an edible pigment can be added as a colorant according to the situation.
(3) The material solution obtained in step (2) is left to stand at 60° C. for stable heat retention for 7 hours to obtain a stable gel solution.
(4) The stable gel solution is cooled to 48° C., dipped using a mold, flipped upward and downward for forming for 6 seconds, and dried in an oven for 150 minutes at 30° C. and RH 50% to obtain a crude product of a capsule cap and a capsule body. The crude product of the capsule cap and the capsule body is released from the mold by a mold release device, cut, and then combined together by sleeving to obtain a hard capsule, that is, a plant-based hard capsule (e.g., a plant-based pharmaceutical hard capsule) for rapid disintegration.

### Embodiment 3

(1) Each raw material is weighted according to the following weight percentages:

| | |
|---|---|
| Hydroxypropyl methylcellulose | 6.41% |
| ι-Carrageenan | 0.30% |
| Gelling gum | 1.53% |
| Potassium chloride | 0.23% |
| Distilled water | 91.53% |

(2) The potassium chloride is dissolved in the distilled water at a normal temperature (e.g., 20° C.-30° C.) and then heated to 40° C. The gelling gum is added, stirred until the gelling gum is evenly dissolved, and then heated to 80° C. The ι-Carrageenan is then added and stirred at a constant temperature of 80° C. for 1 hour until the ι-Carrageenan is completely dissolved. The hydroxypropyl methylcellulose is added and stirred at a constant temperature of 80° C. for 2 hours until the hydroxypropyl methylcellulose is completely dissolved. In this step, an appropriate amount of an edible pigment is added as a colorant according to the situation.
(3) The material solution obtained in step (2) is left to stand at 60° C. for stable heat retention for 6 hours to obtain a stable gel solution.
(4) The stable gel solution is cooled to 40° C., dipped using a mold, flipped upward and downward for forming for 5 seconds, and dried in an oven for 150 minutes at 30° C. and RH 50% to obtain a crude product of a capsule cap and a capsule body. The crude product of the capsule cap and the capsule body is released from the mold by a mold release device, cut, and then combined together by sleeving to obtain a hard capsule, that is, a plant-based hard capsule (e.g., a plant-based pharmaceutical hard capsule) for rapid disintegration.

### Embodiment 4

(1) Each raw material is weighted according to the following weight percentages:

| | |
|---|---|
| Hydroxypropyl methylcellulose | 10.42% |
| ι-Carrageenan | 1.30% |
| Sodium chloride | 0.13% |
| PEG-400 | 1.30% |
| Distilled water | 86.85% |

(2) The sodium chloride is dissolved in the distilled water at a normal temperature (e.g., 20° C.-30° C.) and then heated to 80° C. The ι-Carrageenan is added and stirred at a constant temperature of 80° C. for 1 hour until the ι-Carrageenan is completely dissolved. The hydroxypropyl methylcellulose is added and stirred at a constant temperature of 80° C. for 2.5 hours until the hydroxypropyl methylcellulose is completely dissolved, and the PEG-400 is finally added and stirred to even. In this step, an appropriate amount of an edible pigment can be added as a colorant according to the situation.
(3) The material solution obtained in step (2) is left to stand at 60° C. for stable heat retention for 6 hours to obtain a stable gel solution.
(4) The stable gel solution is cooled to 47° C., dipped using a mold, flipped upward and downward for forming for 6 seconds, and dried in an oven for 150 minutes at 30° C. and RH 50% to obtain a crude product of a capsule cap and a capsule body. The crude product of the capsule cap and the capsule body is released from the mold by a mold release device, cut, and then combined together by sleeving to obtain a hard capsule, that is, a plant-based hard capsule (e.g., a plant-based pharmaceutical hard capsule) for rapid disintegration.

### Embodiment 5

(1) Each raw material is weighted according to the following weight percentages:

| | |
|---|---|
| Hydroxypropyl methylcellulose | 16.39% |
| κ-Carrageenan | 0.41% |
| ι-Carrageenan | 0.41% |
| Glycerin | 0.61% |
| Calcium chloride | 0.20% |
| Potassium chloride | 0.02% |
| Distilled water | 81.96% |

(2) The potassium chloride, the calcium chloride, and the glycerin are dissolved in the distilled water at a normal temperature (e.g., 20° C.-30° C.) and then heated to 80° C. The ι-Carrageenan and the κ-Carrageenan are slowly added in sequence to avoid a formation of agglomerate particles and stirred at a constant temperature of 80° C. for 1.5 hours until the ι-Carrageenan and the κ-Carrageena are completely dissolved. The hydroxypropyl methylcellulose is added and stirred at a constant temperature of 80° C. for 3.5 hours until the hydroxypropyl methylcellulose is completely dissolved to obtain a material solution. In this step, an appropriate amount of an edible pigment is added as a colorant according to the situation;
(3) The material solution obtained in step (2) is left to stand at 60° C. for stable heat retention for 6 hours to obtain a stable gel solution.
(4) The stable gel solution is cooled to 43° C., dipped using a mold, flipped upward and downward for forming for 4 seconds, and dried in an oven for 120 minutes at 30° C. and RH 40% to obtain a crude product of a capsule cap and a capsule body. The crude product of the capsule cap and the capsule body is released from the mold by a mold release device, cut, and then combined together by sleeving to obtain a hard capsule, that is, a plant-based hard capsule (e.g., a plant-based pharmaceutical hard capsule) for rapid disintegration.

### Embodiment 6

(1) Each raw material is weighted according to the following weight percentages:

| | |
|---|---|
| Hydroxypropyl methylcellulose | 14.28% |
| κ-Carrageenan | 0.34% |
| ι-Carrageenan | 0.42% |
| Sorbitol | 0.63% |
| Calcium chloride | 0.29% |
| Potassium citrate | 0.03% |
| Distilled water | 84.01% |

(2) The potassium chloride and the calcium chloride are dissolved in the distilled water at 80° C. and then maintained at 80° C. The ι-Carrageenan and the κ-Carrageenan are slowly added in sequence to avoid a formation of agglomerate particles and stirred at a constant temperature of 80° C. for 1.5 hours until the ι-Carrageenan and the κ-Carrageenan are completely dissolved. The hydroxypropyl methylcellulose is added and stirred at a constant temperature of 80° C. for 3 hours until the hydroxypropyl methylcellulose is completely dissolved. The sorbitol is finally added and stirred to even. In this step, an appropriate amount of an edible pigment is added as a colorant according to the situation.
(3) The material solution obtained in step (2) is left to stand at 60° C. for stable heat retention for 6 hours to obtain a stable gel solution.
(4) The stable gel solution is cooled to 46° C., dipped using a mold, flipped upward and downward for forming for 6 seconds, and dried in an oven for 120 minutes at 30° C. and RH 40% to obtain a crude product of a capsule cap and a capsule body. The crude product of the capsule cap and the capsule body is released from the mold by a mold release device, cut, and then combined together by sleeving to obtain a hard capsule, that is, a plant-based hard capsule (e.g., a plant-based pharmaceutical hard capsule) for rapid disintegration.

### Embodiment 7

(1) Each raw material is weighted according to the following weight percentages:

| | |
|---|---|
| Hydroxypropyl methylcellulose | 6.98% |
| Hydroxypropyl starch | 3.49% |
| ι-Carrageenan | 1.31% |
| Glycerin | 0.87% |
| Sodium chloride | 0.14% |
| Distilled water | 87.21% |

(2) The sodium chloride is dissolved in the distilled water at 80° C. and then maintained at 80° C. The ι-Carrageenan is added and stirred at a constant temperature of 80° C. for 1 hour until the ι-Carrageenan is completely dissolved. The hydroxypropyl starch and the hydroxypropyl methylcellulose are slowly added to avoid a formation of agglomerate particles and stirred at a constant temperature of 80° C. for 180 minutes until the hydroxypropyl starch and the hydroxypropyl methylcellulose are completely dissolved. The glycerin is finally added and stirred to even. In this step, an appropriate amount of an edible pigment is added as a colorant according to the situation.
(3) The material solution obtained in step (2) is left to stand at 60° C. for stable heat retention for 6 hours to obtain a stable gel solution.
(4) The stable gel solution is cooled to 48° C., dipped using a mold, flipped upward and downward for forming for 6 seconds, and dried in an oven for 150 minutes at 30° C. and RH 50% to obtain a crude product of a capsule cap and a capsule body. The crude product of the capsule cap and the capsule body is released from the mold by a mold release device, cut, and then combined together by sleeving to obtain a hard capsule, that is, a plant-based hard capsule (e.g., a plant-based pharmaceutical hard capsule) for rapid disintegration.

### Embodiment 8

(1) Each raw material is weighted according to the following weight percentages:

| | |
|---|---|
| Hydroxypropyl methylcellulose | 8.84% |
| ι-Carrageenan | 0.88% |
| Locust bean gum | 0.44% |
| Sodium chloride | 0.13% |
| PEG-400 | 1.33% |
| Distilled water | 88.38% |

(2) The sodium chloride is dissolved in the distilled water at 80° C. and then maintained at 80° C. The ι-Carrageenan and the locust bean gum are slowly added in sequence to avoid a formation of agglomerate particles and stirred at a constant temperature of 80° C. for 1.5 hours until the ι-Carrageenan and the locust bean gum are completely dissolved. The hydroxypropyl methylcellulose is then added and stirred at a constant temperature of 80° C. for 2 hours until the hydroxypropyl methylcellulose is completely dissolved. The PEG-400 is finally added and stirred to even. In this step, an appropriate amount of an edible pigment is added as a colorant according to the situation.
(3) The material solution obtained in step (2) is left to stand at 60° C. for stable heat retention for 6 hours to obtain a stable gel solution.
(4) The stable gel solution is cooled to 45° C., dipped using a mold, flipped upward and downward for forming for 6 seconds, and dried in an oven for 120 minutes at 30° C. and RH 40% to obtain a crude product of a capsule cap and a capsule body. The crude product of the capsule cap and the capsule body is released from the mold by a mold release device, cut, and then combined together by sleeving to obtain a hard capsule, that is, a plant-based hard capsule (e.g., a plant-based pharmaceutical hard capsule) for rapid disintegration.

### Embodiment 9

(1) Each raw material is weighted according to the following weight percentages:

| | |
|---|---|
| Hydroxypropyl methylcellulose | 8.84% |
| ι-Carrageenan | 1.33% |
| Sodium chloride | 0.14% |
| PEG-4000 | 1.33% |
| Distilled water | 88.36% |

(2) The sodium chloride is dissolved in the distilled water at 80° C. and then maintained at 80° C. The ι-Carrageenan is added and stirred at a constant temperature of 80° C. for 1.5 hours until the ι-Carrageenan is completely dissolved. The hydroxypropyl methylcellulose is added and stirred at a constant temperature of 80° C. for 2 hours until the hydroxypropyl methylcellulose is completely dissolved. The PEG-4000 is finally added and stirred to even. In this step, an appropriate amount of an edible pigment is added as a colorant according to the situation.
(3) The material solution obtained in step (2) is left to stand at 60° C. for stable heat retention for 6 hours to obtain a stable gel solution.
(4) The stable gel solution is cooled to 45° C., dipped using a mold, flipped upward and downward for forming for 6 seconds, and dried in an oven for 120 minutes at 30° C. and RH 40% to obtain a crude product of a capsule cap and a capsule body. The crude product of the capsule cap and the capsule body is released from the mold by a mold release device, cut, and then combined together by sleeving to obtain a hard capsule, that is, a plant-based hard capsule (e.g., a plant-based pharmaceutical hard capsule) for rapid disintegration.

### Embodiment 10

(1) Each raw material is weighted according to the following weight percentages:

| | |
|---|---|
| Hydroxypropyl methylcellulose | 9.63% |
| ι-Carrageenan | 1.31% |
| Sodium chloride | 0.13% |
| Calcium chloride | 0.04% |
| Glycerin | 1.31% |
| Distilled water | 87.58% |

(2) The sodium chloride and the calcium chloride are dissolved in the distilled water at 80° C. and then maintained at 80° C. The ι-Carrageenan is added and stirred at a constant temperature of 80° C. for 2 hours until the ι-Carrageenan is completely dissolved. The hydroxypropyl methylcellulose is added and stirred at a constant temperature of 80° C. for 2 hours until the hydroxypropyl methylcellulose is completely dissolved. The glycerin is finally added and stirred to even. In this step, an appropriate amount of an edible pigment is added as a colorant according to the situation;
(3) The material solution obtained in step (2) is left to stand at 60° C. for stable heat retention for 6 hours to obtain a stable gel solution.
(4) The stable gel solution is maintained at 60° C., dipped using a mold, flipped upward and downward for forming for 6 seconds, and dried in an oven for 120 minutes at 30° C. and RH 40% to obtain a crude product of a capsule cap. The crude product of the capsule cap and the capsule body is released from the mold by a mold release device, cut, and then combined together by sleeving to obtain a hard capsule, that is, a plant-based hard capsule (e.g., a plant-based pharmaceutical hard capsule) for rapid disintegration.

It should be noted that specific parameters or some common reagents in the aforementioned embodiments are specific embodiments or preferred embodiments under a conception of the present disclosure, rather than a limitation of the present disclosure. A person of ordinary skill in the art can perform adaptive adjustments within the conception and a scope of the present disclosure. In addition, unless otherwise specified, raw materials used in the present disclosure can also be conventional commercial products in the art or prepared by conventional methods in the art.

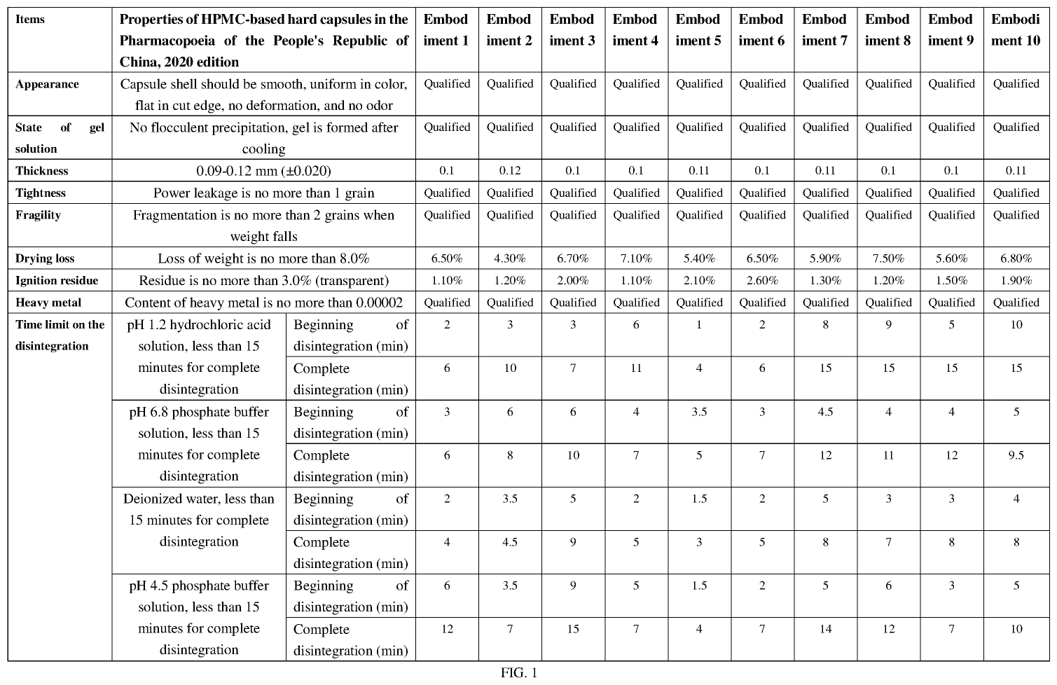

The plant-based hard capsules for the rapid disintegration prepared in Embodiments 1-10 are tested according to properties of hydroxypropyl methylene cellulose based (HPMC-based) hard capsules in the Pharmacopoeia of the People's Republic of China, 2020 edition, and specific test results are shown in FIG. 1.

The aforementioned test items of the HPMC-based hard capsules focus on the plant-based hard capsules for rapid disintegration prepared by the present disclosure, and a disintegration rate is greatly improved on a premise that physical-chemical properties of the capsules are ensured. A disintegration time is tested by a disintegration test conducted in four different media after filling with talc powder. Specifically, the disintegration time is tested by a disintegration analyzer of Tianda Tianfa. In the import registration standard for HPMC-based hard capsules (JX20050073), it is required that capsules should be completely disintegrated within 15 minutes. After a test, it is found that a disintegration time of the plant-based hard capsules for the rapid disintegration prepared by the present disclosure is basically within 10 minutes, and some of the plant-based hard capsules for the rapid disintegration can even reach 5 minutes or less.

It can also be seen from the test results of the embodiments 1-10 and FIG. 1 that a problem of a long disintegration time and slow dissolution rate of the HPMC-based hard capsules caused by adding carrageenan as a gel-forming agent is solved by compounding carrageenan and inorganic salt ions (i.e., cations and anions). Specifically, in the embodiments 1-10 of the present disclosure, the cations are mainly K⁺, Na⁺, and Ca²⁺, and ι-carrageenan interacts with K⁺, Na⁺, and Ca²⁺. In the embodiments of the present disclosure, the anions are mainly Cl⁻ and citrate ion. A gel formed by Cl⁻ and two types of carrageenan has faster disintegration performance, while the gel formed by the citrate ion and two types of carrageenan has greater strength and faster film formation rate.

A uniformity of a thickness of a single wall is an important property to ensure a uniformity and a stability of the capsule. Only when the capsules have better uniformity, will an on-machine rate of the plant-based hard capsules be high and the disintegration time of the plant-based hard capsules be accelerated. After a pilot production, the plant-based hard capsules for the rapid disintegration prepared by the present disclosure has uniform and controllable wall thickness and has potential for industrial production.

In addition, a person of ordinary skill in the art should understand that although there are many problems in the prior art, each of embodiments 1-10 or each of the technical solutions of the present disclosure can be only improved in one or several aspects, rather than solving all technical problems listed in the prior art or the background at the same time. A person of ordinary skill in the art should understand that content not mentioned in a claim should not be used as a limitation of the claim.

Although many terms, such as a coagulation aid, a gel-forming agent, a film formation agent, plasticizer, etc. are used in the specification, a possibility of using other terms is not excluded. The terms are only used to more conveniently describe and explain a substance of the present disclosure. The terms should not be interpreted as any additional limitation that is contrary to the spirit of the present disclosure. The terms “first”, “second”, and the like (if any) in the description and claims of the embodiments of the present disclosure and the above drawings are used to distinguish similar objects and need not be used to describe a specific priority or a specific sequence.

Finally, it should be noted that the aforementioned embodiments are only used to illustrate the technical solutions of the present disclosure, rather than a limit of the technical solutions of the present disclosure. Although the present disclosure is described in detail with reference to the embodiments, a person of ordinary skill in the art should understand that the technical solutions described in the embodiments can still be modified, some or all of the technical features can be equally replaced, while these modifications or replacements will not cause the substance of the technical solutions to depart from the scope of the technical solutions of the embodiments of the present disclosure.

What is claimed is:

1. A method for preparing a plant-based hard capsule for rapid disintegration, comprising:
(1) weighing coagulant aids with a weight percentage of 0.01%-3%, dissolving in distilled water at 75-90° C., and stirring until the coagulant aids are completely dissolved to obtain a first solution; maintaining a temperature of the first solution at 75-90° C., adding a gel-forming agent with a weight percentage of 0.1%-5%, and stirring until the gel-forming agent is completely dissolved; adding a film-forming agent with a weight percentage of 5%-25%, stirring for 2-5 hours, finally adding a plasticizer with a weight percentage of 0-3%, and stirring until the film-forming agent and the plasticizer are completely dissolved to obtain a material solution;
(2) leaving the material solution obtained in the step (1) to stand at 45-60° C. for 8-12 hours to obtain a stable gel solution; and
(3) cooling the stable gel solution obtained in the step (2) to 40-50° C., dipping into the stable gel solution for forming, then drying for 100-200 minutes under a condition of 20-35° C. and a 40%-60% relative humidity to obtain a crude product of a capsule cap and a capsule body; and demoulding, cutting, and combining together by sleeving to obtain the plant-based hard capsule for the rapid disintegration.

2. The method according to claim 1, wherein the film-forming agent comprises at least one of hydroxypropyl methylcellulose (HPMC), sodium carboxymethyl starch, or hydroxypropyl starch.

3. The method according to claim 1, wherein the gel-forming agent comprises at least one of Gelan gum, ι-Carrageenan, or κ-carrageenan.

4. The method according to claim 1, wherein the coagulant aids comprise at least one of potassium citrate, sodium citrate, potassium chloride, sodium chloride, or calcium chloride.

5. The method according to claim 1, wherein the plasticizer comprises at least one of glycerol, polyethylene glycol (PEG), or sorbitol.

6. The method according to claim 1, wherein the gel-forming agent is ι-Carrageenan or κ-Carrageenan.

7. The method according to claim 6, wherein the coagulant aids comprise potassium chloride or potassium citrate and at least one of sodium citrate, sodium chloride, or calcium chloride.

8. The method according to claim 7, wherein the film-forming agent is hydroxypropyl methylcellulose.

9. The method according to claim 1, wherein a weight ratio of the film-forming agent, the gel-forming agent, the coagulant aid, the plasticizer, and the distilled water is (6.41-16.39):(0.76-1.83):(0.13-0.31):(0-1.33):(81.95-91.53).

10. A plant-based hard capsule for rapid disintegration prepared by the method according to claim 1.

11. A plant-based hard capsule for rapid disintegration prepared by the method according to claim 2.

12. A plant-based hard capsule for rapid disintegration prepared by the method according to claim 3.

13. A plant-based hard capsule for rapid disintegration prepared by the method according to claim 4.

14. A plant-based hard capsule for rapid disintegration prepared by the method according to claim 5.

15. A plant-based hard capsule for rapid disintegration prepared by the method according to claim 6.

16. A plant-based hard capsule for rapid disintegration prepared by the method according to claim 7.

17. A plant-based hard capsule for rapid disintegration prepared by the method according to claim 8.

18. A plant-based hard capsule for rapid disintegration prepared by the method according to claim 9.
